(19) United States
(12) Patent Application Publication
(10) Pub. No.: US 2009/0301346 A1
(43) Pub. Date: Dec. 10, 2009

(54) DENTAL RESIN-BASED CEMENT COMPOSITION

(76) Inventors: Hisaki Tanaka, Kyoto (JP); Toshihide Fujii, Kyoto (JP); Yoshiaki Kohro, Kyoto (JP); Keisuke Torii, Kyoto (JP); Mikito Deguchi, Kyoto (JP)

Correspondence Address: WENDEROTH, LIND & PONACK, L.L.P., 1030 15th Street, N.W.,, Suite 400 East, Washington, DC, 20005-1503, US

(21) Appl. No.: 12/448,049

(22) PCT Filed: Dec. 6, 2006
(86) PCT No.: PCT/JP2006/324354

§ 371 Date: Jun. 5, 2009

(51) Int. Cl. A61K 6/02

(52) U.S. Cl. 106/35

(57) ABSTRACT

The present invention provides a dental resin cement composition comprising at least two kinds of pastes, one kind of a paste comprising (a) 20 to 50 parts by weight of a polymerizable monomer composition and (b) 50 to 80 parts by weight of a filler consisting of an inorganic compound, or an organic composite comprising an inorganic compound, amounts of (a) and (b) being based on conversion of 100 parts by weight of the dental resin-based cement composition paste, and the filler (b) comprising (c) 2.0 to 10.0 parts by weight of fine silica particles based on the above conversion, and having (d) a yield viscosity of from 100 to 3,000 [Pa·s], and (e) a thixotropy index of 3.0 or more; and another kind of a paste having the same constituent features (a) to (c) and (e) except that the paste has (d) a yield viscosity of from 70 to 4,000 [Pa·s], wherein upon use, the at least two kinds of pastes are used by mixing them.

### BACKGROUND OF THE INVENTION

### Field of the Invention

The present invention relates to a dental resin-based cement composition having excellent operability, and a method for controlling flow properties.

Effectiveness of a dental cement composition having good operability lies in shortening of the operation time and reduction of technical errors, which account for the majority of user demands.

Furthermore, according to the method for controlling flow properties of the present invention, it is easy to considerably vary flow properties of a pasty composition and thus improvement in operability is accomplished.

In the dental field, a dental cement is used for various purposes such as luting, temporary bonding, filling, temporary sealing and relining. Among them, a cement for luting is used to attach prostheses such as crowns and bridges, or restorations such as inlays and onlays when the tooth is fractured or degraded by tooth decay.

The thickness of a cement existing between dentin and metal (prostheses, restoration) exerts an influence on the luting strength or bonding strength, or solubility of the cement. The magnitude of the thickness is called coating thickness and is influenced by the temperature, powder/liquid ratio, kneading time, kneading method, particle size or the like. Conditions suited for use as the dental cement include (1) no irritation of tooth pulp, (2) good operability, (3) high strength after solidification, (4) less dissolution in saliva, (5) bonding force to dentin, and (6) color similar to dentin.

A dental resin-based cement composed of a paste having excellent properties with respect to bonding force, strength after solidification, tooth pulp irritation or the like has been developed in recent years and is widely used in clinical fields which particularly require a bonding force. As used herein, the dental resin-based cement is a cement which is prepared by mixing at least two pastes each having the same or different composition and contains a polymerizable monomer as the constituent component, and also had a problem such as poor operability of a paste used in the cement because of flow properties caused by the polymerizable monomer.

A commercially available dental resin-based cement composition is a dental bonding resin cement composed of two pastes and is also excellent in paste viscosity after kneading, collection and sagging property of a kneaded paste using a spatula, and operability such as applicability of the paste to prostheses. However, it is inferior in paste miscibility and is also inferior in kneadability since two pastes are quite different in fluidity. Since both pastes are inferior in miscibility at an initial stage of kneading, when using a container equipped with mixing elements at a paste ejecting site, namely, an automix container, it was sometimes impossible to automatically carry out a sufficient kneading operation in a chip having mixing elements of a container tip.

Another commercially available dental resin-based cement composition is a dental bonding resin cement composed of two pastes and easily carries out an initial kneading operation and is excellent in miscibility since two pastes have very similar fluidities. However, it does not exhibit thixotropic nature because of its low viscosity, and the paste during kneading exhibits high paste viscosity and strong stringiness. Since the kneaded paste is likely to be sagged by a spatula and is not excellent in operability. Furthermore, when the automix container is used, miscibility in the chip having mixing elements of a container tip is excellent, but sag of the paste is likely to be caused by ejection delay of the paste from the chip tip.

Another commercially available dental resin-based cement composition is a dual curing type dental bonding resin cement composed of two pastes and a user is under stress since it is inferior in miscibility between pastes, and high viscosity and high stringiness of the paste upon kneading. Since the paste is likely to cause sag and it is difficult to carry out the operation using a spatula, it cannot be said that the composition has excellent operability suited for use as a cement.

Japanese Patent Application Publication (JP-A) No. 2002-514211 discloses, as a dental composition having improved handling performances, rheological conditions having excellent operability in a dental composition with respect to a specific composition. However, the conditions are conditions for a restoring filler material and definition of operability is different when compared with dental cement.

The excellent handling property of a restoring filler material in Japanese Patent Application Publication (JP-A) No. 2002-514211 is that it does not exhibit slump and easily adapts to a specimen cavity, and is easily subjected to feathering since it is easily contoured, and also it does not adhere to an indwelling apparatus and can be generally used for restoration of a tooth structure quickly and easily. “Slump” means a phenomenon in which the material flows as a result of an action of gravity. Since the dentist desires that the material retains a shape until the material is cured after indwelling in the mouth and further contouring, it is preferred that the restoring filler of the tooth does not cause slump. The cement composition in the present invention is a bonding material and does not require an operation of indwelling in the mouth and further contouring and therefore the operability is different from the operability imparted by the above publication. “Contouring” means the step of forming a restoring filler material using a dental apparatus so that the restoring filler material has a structure similar to the natural tooth. To facilitate contouring, the material must have sufficient high viscosity to be able to retain the shape after operating using a dental apparatus. The cement composition in the present invention is a bonding material and does not require the step of forming the restoring filler material using a dental apparatus. Therefore, the operability is different from the operability imparted by the publication. “Feathering” means the step of forming the material into a thin film so as to make the restoring filler material fit the state of the natural tooth, and the cement composition in the present invention is a bonding material and the operability is different from that imparted by the publication.

Japanese Patent Application Publication (JP-A) No. 2001-510146 discloses a dental resin cement composition having improved handling properties and rheological conditions having excellent operability, but is a resin dental cement material with respect to a specific composition containing a polymer as a handling properties improver and is different from that of the present invention.

Since the polymer acting as a handling properties improver is not dispersed in a preferred resin system which has conventionally been used in a dental material, sufficient strength and durability required for the dental material cannot be obtained.

Furthermore, use of the polymer is not preferred since the amount of the inorganic filler filled is finally decreased, lending to deterioration of preferred properties depending on the inorganic filler, for example, X-ray opaqueness, fluorine sustained releasability, high elastic modulus, transparency and the like.

The dental resin-based cement is generally provided as a plurality of pastes and is kneaded so as to form into a final product. Such a kneading process exerts an adverse influence on physical properties of a cured article as a result of an influence of entrainment of bubbles, and also the operation is remarkably troublesome.

Therefore, when a supply container using an automix chip disclosed in U.S. Pat. No. 6,820,766 is used, a plurality of pastes must be mixed in the same amount in the automix chip.

However, the dental resin-based cement is not mixed in the same amount because of non-uniform values for the rheological properties of a plurality of pastes, and thus a considerable adverse influence tends to exert on properties of the product. In contrast, a dental resin-based cement, which is easily mixed in the same amount, may cause considerable deterioration of operation properties such as “sag” and “flow resistance”.

Therefore, an improvement in operability of the dental resin-based cement is demanded by users.

Furthermore, a method for controlling flow properties of a paste, in which more easy improvement in operability can be expected, is desired.

### SUMMARY OF THE INVENTION

An object of the present invention is to provide a dental resin-based cement composition having good operability, and a method for controlling flow properties. A dental resin-based cement containing a polymerizable monomer as a base material has poor operability and had a clinical problem in view of operation time and technical errors.

As used herein, the term “operability” means properties with respect to cement nature and sensibly means kneading operability, fluidity of a kneaded mixture or the like. In general, these properties can be controlled by constitution contents of the filler, for example, particle composition, particle shape, particle diameter, amount of particle filled, particle surface treating agent, particle surface treating method, and resin additives.

However, variation of the constitution of the filler component in the paste composition may considerably deteriorate mechanical properties and optical properties of a cement cured article as a final form.

Namely, the operability of the paste composition means those characteristics which easily vary with the conditions such as components and amounts. Therefore, there have been desired controlling methods and compositions which can vary flow properties of the dental resin-based cement composition easily and considerably.

However, when the technical scope required to satisfy performances required for good operability is expressed, since it is difficult to express by existing prescripts such as components and amounts, it is appropriate to express by flow properties of the material.

As used herein, the term “operability” in the dental cement paste means properties with respect to paste nature and can be sensibly expressed as values of characteristics such as kneading operability, fluidity of a kneaded mixture or the like.

The values of required characteristics with respect to the operability are more specifically “admixing”, “kneading resistance”, “sagging” and “flow resistance”.

The term “admixing” means operability when slightly mixing at an initial stage of kneading in case where a dental cement provided as a semifinished product is kneaded to form a finished product, and it is necessary that mixability of a powder and powder, or a paste and a paste is excellent.

Generally, a dental cement must be strongly kneaded on a cement kneading board using a spatula and this kneading operation is called kneading. The term “kneading resistance” means resistance of a cement powder/liquid or a cement paste during kneading. It is necessary that kneading resistance is small.

The term “sagging” means sagging from a spatula when the kneaded cement paste is then applied to or filled in a restoration using the spatula. It is necessary that sagging does not occur.

The term “flow resistance” means a resistance feeling such that when the luting operation to the site to be attached is carried out after application of the cement to prostheses or filling of the cement in prostheses, a cement paste is fluidized, and thus prostheses are smoothly luted and the cement paste reaches to corners and borders of prostheses having a complicated form without the lack of the cement paste.

Effectiveness of a dental cement composition having good operability lies in shortening of the operation time and reduction of technical errors, which account for the majority of dentist's demands. Although mechanical properties and bonding properties of the dental cement have been intensively studied, there have been few reports on good operability and the problem is still to be solved.

Various properties with respect to operability achieved by satisfactory control of the constitution of the composition have a correlation with the flow properties of the material. As used herein, the term “flow properties of the material, which have a correlation with various properties with respect to the operability” are yield viscosity and thixotropy index of the paste. The cement material is generally a “sol” which is a particle dispersed composition containing a liquid dispersion medium. In order to obtain good evaluation about “sagging” property as properties with respect to the operability, the cement material should be in a “gel” state where fluidity is lost even if it is a “sol”.

However, regarding “admixing” and “flow resistance”, the cement material should be in a “sol” state with higher fluidity.

Therefore, in order to obtain good operability about “sagging” property, it is important to achieve a “gel” state required as the cement material and this is expressed by yield viscosity. Furthermore, regarding “admixing” and “flow resistance”, it is important that transformation from the “gel” state to the “sol” state or transformation from the “sol” state to the “gel” state is sensibly clear, and this is expressed by a thixotropy index.

Since the dental resin-based cement is generally provided as a plurality of pastes and contains a polymerization initiator in the paste so as to form into a final product in clinical practice, it is difficult to evaluate the flow properties of the material.

However, it is easy to evaluate the flow properties of individual paste and, even if the final form is a plurality of pastes, it is possible to estimate from evaluation of flow properties of individual paste. Even when a supply container using an automix chip having good operability is used, a plurality of pastes should be sufficiently kneaded in the same amount in the automix chip.

The present inventors have intensively studied so as to achieve the above object and found out that a dental resin-based cement composition having excellent operability and a method for controlling flow properties of the same can be obtained by inclusion of at least two kinds of pastes, each paste containing a specific amount of a polymerizable monomer composition, a specific amount of a filler composed of an inorganic compound, or an organic composite containing the inorganic compound and a specific amount of fine silica particles, and having specific yield viscosity and thixotropy index, wherein at least two kinds of pastes are used by mixing them upon use. Thus, the present invention has been completed.

The present invention provides the followings.

(1) A dental resin cement composition comprising at least two kinds of pastes, one kind of a paste comprising (a) 20 to 50 parts by weight of a polymerizable monomer composition and (b) 50 to 80 parts by weight of a filler consisting of an inorganic compound, or an organic composite comprising an inorganic compound, amounts of (a) and (b) being based on conversion of 100 parts by weight of the dental resin-based cement composition paste, and the filler (b) comprising (c) 2.0 to 10.0 parts by weight of fine silica particles based on the above conversion, and having (d) a yield viscosity of from 100 to 3,000 [Pa·s], and (e) a thixotropy index of 3.0 or more; and another kind of a paste having the same constituent features (a) to (c) and (e) except that the paste has (d) a yield viscosity of from 70 to 4,000 [Pa·s], wherein upon use, the at least two kinds of pastes are used by mixing them.
(2) The dental resin-based cement composition according to (1), wherein the composition comprises at least two kinds of pastes, each of the pastes comprising (a) 20 to 50 parts by weight of a polymerizable monomer composition and (b) 50 to 80 parts by weight of a filler consisting of an inorganic compound, or an organic composite comprising an inorganic compound, amounts of (a) and (b) being based on conversion of 100 parts by weight of the dental resin-based cement composition paste, and the filler (b) comprising (c) 2.0 to 10.0 parts by weight of fine silica particles based on the above conversion, and having (d) a yield viscosity of from 100 to 3,000 [Pa·s], and (e) a thixotropy index of 3.0 or more, and wherein upon use, the at least two kinds of pastes are used by mixing them.
(3) The dental resin-based cement composition according to (1) or (2), wherein the composition further includes a characteristic (f) in the two kinds of pastes according to (1), letting the paste having the smaller yield viscosity (d) to be a first paste, and the other paste to be a second paste, 20 viscosity values are determined by using 20 measurement points within the range from 0.1 [s⁻¹] to 10 [s⁻¹] so as to include shear rates 0.1 [s⁻¹] and 10 [s⁻¹] and to make the intervals between logarithms of the shear rates equal, and when a proportionality constant β and a determination coefficient R² of a linear approximation curve passing through the origin are calculated with respect to a function in which 20 viscosity values of the first paste are independent variables and 20 viscosity values of the second paste are dependent variables, β is within the range from 1.00 to 15.00 and R² is within the range from 0.50 to 1.00.
(4) A method for controlling flow properties of a dental resin-based cement composition, which comprises mixing at least two kinds of pastes, each of the pastes comprising (a) 20 to 50 parts by weight of a polymerizable monomer composition and (b) 50 to 80 parts by weight of a filler consisting of an inorganic compound or an organic composite comprising an inorganic compound, amounts of (a) and (b) being based on conversion of 100 parts by weight of the dental resin-based cement composition paste, and the filler (b) comprising (c) 2.0 to 10.0 parts by weight of fine silica particles based on the above conversion, and using the mixture as a dental resin-based cement composition,

wherein upon use, one kind of the pastes having (d) a yield viscosity of 100 to 3,000 [Pa·s] and (e) a thixotropy index of 3.0 or more, and another kind of the pastes having (d) a yield viscosity of 70 to 4,000 [Pa·s], and the same (e) as that described above are selected.

(5) The method according to (4), wherein the method comprises mixing at least two kinds of pastes, each comprising (a) 20 to 50 parts by weight of a polymerizable monomer composition and (b) 50 to 80 parts by weight of a filler consisting of an inorganic compound, or an organic composite comprising an inorganic compound, amounts of (a) and (b) being based on conversion of 100 parts by weight of a dental resin-based cement composition, and the (b) filler comprising (c) 2.0 to 10.0 parts by weight of silica fine particles, and using the mixture as a dental resin-based cement composition,

where upon use, the at least two kinds of pastes, each having (d) a yield viscosity of 100 to 3,000 [Pa·s] and (e) a thixotropy index of 3.0 or more, are selected.

(6) The method according to (4) or (5), wherein as the two kinds of pastes as defined in (5), the two kinds of pastes are selected so that (f) letting the paste having the smaller yield viscosity (d) to be a first paste and the other paste to be a second paste, 20 viscosity values are determined by using 20 measurement points within the range from 0.1 [s⁻¹] to 10 [s⁻¹] so as to include shear rates 0.1 [s⁻¹] and 10 [s⁻¹] and to make the intervals between logarithms of the shear rates equal, and when a proportionality constant β and a determination coefficient R² of a linear approximation curve through the origin are calculated with respect to a function in which 20 viscosity values of the first paste are independent variables and 20 viscosity values of the second paste are dependent variables, β is within the range from 1.00 to 15.00 and R² is within the range from 0.50 to 1.00.

The method of measuring and the method of calculating a yield viscosity of the paste and a thixotropy index of the paste in the present invention are described.

1) A rotary rheometer is used.
2) A cone-flat plate jig in which an angle of a sample interval is 4° is used.
3) Measurement is carried out under an environment at a temperature of 23° C. under an atmospheric pressure.
4) A steady flow viscosity at a shear rate of 0.1 to 10 [s⁻¹] is measured.
5) A measurement interval is determined by using 20 measurement points within the range from 0.1 [s⁻¹] to 10 [s⁻¹] so as to include shear rates 0.1 [s⁻¹] and 10 [s⁻¹] and to make the intervals between logarithms of the shear rates equal.
6) In order to make stress history of a measurement sample constant, 10 seconds after applying a shear rate of 10 [s⁻¹] for 60 seconds, measurement is initiated.

Herein, a steady flow viscosity at a shear rate of 0.1 [s⁻¹] is taken as a yield viscosity of the paste. The thixotropy index is given by Equation (1) where ρ_{0.1} [Pa·s] denotes a viscosity at a shear rate of 0.1 [s⁻¹] and ρ₁₀ [Pa·s] denotes a viscosity at a shear rate of 10 [s⁻¹].

Thixotropy index=ρ_{0.1}/ρ₁₀ Equation (1)

Furthermore, there is provided a dental resin-based cement composition in which n kinds or more (wherein n is an integer of 2 or more) of pastes are cured by a chemical reaction after mixing.

### EFFECT OF THE INVENTION

The dental resin-based cement composition of the present invention has excellent operability which satisfies required properties such as “admixing” “kneading resistance”, “sagging” and “flow resistance” required by a clinical practice.

Particularly, “admixing” properties of mixability of mixing pastes were improved. When kneading properties are inferior, mixing takes a long time and the working time is prolonged, and thus a burden on patients and doctors increases and a stress increases. This stress can cause technical errors.

When the kneading work was not sufficiently carried out because of inappropriate “admixing” properties, it is difficult to obtain mechanical properties and high bonding properties. Furthermore, bubbles are easily entrained into the paste, thus making it difficult to remove bubbles. As a result, it becomes impossible to obtain high mechanical properties and high bonding properties as characteristics of the dental resin-based cement.

The mixing method include a method in which a kneading work is carried out on a kneading paper using a spatula and a method in which a container equipped with mixing elements, namely, an automix container is used. In the case of the cement using the automix container, the kneading work is automatically carried out in a chip having mixing elements at a container tip. Since the above kneading work is not carried out, kneading must be securely carried out in the chip. A dental cement having good kneadability is desired so as to avoid failure of kneading.

In addition to “admixing” properties, “kneading resistance” properties are important during the kneading work. Herein, “kneading resistance” properties have a correlation with resistance of the paste during the kneading work. When “kneading resistance” properties are inferior, a feeling of a high viscosity of the paste gives a stress to the worker and also the stringiness is too strong to knead. Therefore, mixing takes a long time and the working time is prolonged, and thus a burden on patients and doctors increases and a stress increases. A stress on doctors can cause technical errors.

Next, the luting operation to the site to be attached is carried out after application of the cement to prostheses or filling of the cement in prostheses. In this case, “flow resistance” properties are important. The term “flow resistance” properties mean properties which enable a cement paste to flow when the luting operation to the site to be attached is carried out after application of the cement to prostheses or filling of the cement in prostheses, and thus prostheses are smoothly luted and the cement paste reaches to corners and borders of prostheses having a complicated form without the lack of the cement paste. Because of inappropriate “flow resistance” properties, there is a large possibility to cause floating of prostheses. Floating of prostheses causes a large loss on patients and there is a possibility of causing not only adverse effects at an initial stage of a treatment with respect to esthetics and incompatible occlusion, but also long-term adverse effects that can cause secondary dental caries and deterioration of prostheses.

Furthermore, the kneaded cement paste is applied to prostheses or filled in the prostheses using a spatula. In that case, it is necessary that the kneaded cement paste does not sag from the spatula. Herein, “sagging” properties in the present invention are important. It becomes difficult for doctors to remove the excess cement after wearing of prostheses because of inappropriate “sagging” properties, leading to a long working time and a stress. In the case of removing the excess cement, it is necessary that the cement forms a mass without causing sagging. There is a risk for patients that the unpolymerized resin component is contacted with oral mucosa as a result of sagging of the paste in the oral cavity because of inappropriate “sagging” properties, thus causing allergy of patients.

In the method for controlling the dental resin-based cement composition of the present invention, it is easy to carry out an improvement in flow properties of the paste, which has a correlation with an improvement in operability.

### BEST MODE FOR CARRYING OUT THE INVENTION

As the polymerizable monomer composition (a), for example, a monofunctional or polyfunctional polymerizable monomer, which is usually used as a dental composition, can be used.

Among these, as the monofunctional polymerizable monomer, for example, hydrocarbon esters of methacrylic acid, such as methyl methacrylate, ethyl methacrylate, butyl methacrylate and hexyl methacrylate, and acrylates corresponding to these methacrylates, preferably methyl methacrylate are used.

As the monofunctional polymerizable monomer having a hydroxyl group, for example, 2-hydroxyethyl methacrylate, 2 or 3-hydroxypropyl methacrylate, 4-hydroxybutyl methacrylate, diethylene glycol monomethacrylate, dipropylene glycol monomethacrylate, glycidyl methacrylate, tetrahydrofurfuryl methacrylate and allyl methacrylate, and acrylates corresponding to these methacrylates, preferably 2-hydroxyethyl methacrylate are used.

As the polyfunctional polymerizable monomer, difunctional monomers, for example, polymethacrylates of alkanepolyol, such as ethylene glycol dimethacrylate, propylene glycol dimethacrylate and neopentyl glycol dimethacrylate, and acrylates corresponding to these methacrylates, preferably ethylene glycol dimethacrylate are used.

As the polyoxyalkanepolyol polymethacrylate, for example, diethylene glycol dimethacrylate, triethylene glycol dimethacrylate and dipropylene glycol dimethacrylate, and acrylates corresponding to these methacrylates, preferably triethylene glycol dimethacrylate are used. Alternatively, difunctional methacrylates having a urethane bond obtained by the addition reaction of 1 mol of a diisocyanate compound and 2 mol of a hydroxyl group-containing methacrylate such as 2-hydroxyethyl methacrylate, and acrylates corresponding to these methacrylates, and

2,2-bis(4-(2-hydroxy-3-methacryloxypropoxy)phenyl)propane (bis-GMA), 2,2-bis-(4-methacryloyloxyphenyl)propane, 2,2-bis-(4-methacryloyloxypolyethoxyphenyl)propane (D-2.6E), 2,2-bis(4-methacryloyloxydiethoxyphenyl)propane, 2,2-bis(4-methacryloyloxytetraethoxyphenyl)propane, 2,2-bis(4-methacryloyloxypentaethoxyphenyl)propane and 2,2-bis(4-methacryloyloxydipropoxyphenyl)propane, and acrylates corresponding to these methacrylates, preferably 2,2-bis(4-(2-hydroxy-3-methacryloxypropoxy)phenyl)propane (bis-GMA) are used.

As the trifunctional monomer, for example, trimethylolpropane trimethacrylate, trimethylolethane trimethacrylate, pentaerythritol trimethacrylate and trimethylolmethane trimethacrylate, and acrylates corresponding to these methacrylates, preferably trimethylolpropane trimethacrylate are used.

As the tetrafunctional monomer, for example, pentaerythritol tetramethacrylate and tetrafunctional urethane methacrylate, and acrylates corresponding to these methacrylates are used.

A pentafunctional or higher functional monomer may also be used.

These monofunctional or polyfunctional polymerizable monomers can be used alone, or two or more kinds of them can be used in combination.

Furthermore, as the filler component (b), for example, fillers used usually in the dental composite material can be used.

Examples of the inorganic filler include silica, aluminum silicate, alumina, zirconium silicate, zirconia, titania, various glasses (including fluoride glass, borosilicate glass, soda glass, barium glass, barium aluminum silica glass, glass containing strontium or zirconium, glass ceramics, fluoroaluminosilicate glass, and synthetic glass obtained by a sol-gel method), Aerogil®, calcium fluoride, strontium fluoride, calcium carbonate, kaolin, clay, mica, aluminum sulfate, calcium sulfonate, barium sulfate, titanium oxide, calcium phosphate, hydroxyapatite, calcium hydroxide, strontium hydroxide and zeolite. The inorganic filler further includes agglomerated particles of a plurality of inorganic oxides, for example, composite inorganic oxide particles disclosed in Japanese Patent Publication (Laid-Open (JP-A)) No. 2001-302429.

Examples of the organic filler include polymethyl methacrylate (PMMA), polyethyl methacrylate, polypropyl methacrylate, polybutyl methacrylate, polyvinyl acetate, polyethylene glycol, polypropylene glycol and polyvinyl alcohol. Examples of the organic composite filler include those obtained by polymerization-coating the surface of the inorganic filler with a compound exemplified as the polymerizable monomer and grinding into particles having an appropriate particle diameter, and particles obtained by preliminarily mixing the polymerizable monomer with the inorganic filler and subjecting to the operation such as emulsion polymerization or suspension polymerization.

These fillers are preferably subjected to surface treatment with the known titanate coupling agent, aluminate coupling agent or silane coupling agent. Examples of the silane coupling agent include γ-methacryloxypropyltrimethoxysilane and γ-methacryloxypropyltriethoxysilane. Preferably, γ-methacryloxypropyltrimethoxysilane is used.

The amount of the filler component (b) used in the present invention is within the range from 50 to 80 parts by weight, and preferably from 55 to 75 parts by weight, based on the paste.

When the amount of the filler component (b) used in the present invention is less than 50 parts by weight since high strength, high elasticity, high X-ray opaqueness, low polymerization shrinkage and fluorine sustained releasability required as the dental cement material are insufficient, being inappropriate.

When the amount of the filler component (b) used in the present invention is more than 80 parts by weight since a viscosity of the paste is very high and it is difficult to obtain good operability, being inappropriate.

The filler used in the present invention contains fine silica particles (c) and the fine silica particles have a primary particle average particle size of 0.1 to 100 nm, and hydrophobic fine silica particles having a primary particle average particle size of 10 to 50 nm are preferred.

These hydrophobic fine silica particles mean fine silica powders surface-treated with a silane coupling agent and/or a modified silicone oil.

In order to further improve hydrophobicity and to improve thickening properties and thixotropic properties of the adhesive resin, fine silica powders may be treated with a hydrophobicity improver after or at the same time with a treatment with the silane coupling agent and/or modified silicone oil. The hydrophobicity improver which can be used for the treatment is an organosilicon compound which reacts with fine silica powders or physically sorb onto fine silica powders, and examples thereof include hexamethyldisilazane, dimethylpolysiloxane, methylchlorosilane, alkyltrialkoxysilane and dialkyldialkoxysilane.

Examples of the fine silica powders include dry silica, silica aerogel and wet silica, of which dry silica is more preferred.

Among these fine silica powders, dry silica is commercially available from NIPPON AEROSIL CO., LTD. under the trade name of Aerosil. These fine silica powders may be, for example, Aerosil 50, Aerosil 90, Aerosil 130, Aerosil 200, Aerosil 300, Aerosil 380, Aerosil OX50 and Aerosil TT600, and fine silica powders whose surface is subjected to hydrophobization treatment, such as Aerosil R972, Aerosil R974, Aerosil R976, Aerosil R976S, Aerosil R202, Aerosil R812, Aerosil R812S, Aerosil R805, Aerosil R104, Aerosil R106, RY200, RX200, R711, RY200S, RA200H, R8200 and RA200HS. Furthermore, silica powders manufactured by Cabot (USA) Corporation under the trade name of Cab-O-Sil and silica powders manufactured by WACKER CHEM GMBH under the trade name of HDK can also be used.

Regarding the amount of components of fine silica particles used in the present invention, the amount of the component of fine silica particles contained in the pasty composition is within the range from 2.0 to 10.0 parts by weight and the fine silica particles are appropriately blended so that the resultant paste has a yield viscosity of 100 to 3,000 [Pa·s] and the resultant paste has a thixotropy index of 3.0 or more.

The dental resin-based cement composition in the present invention contains a polymerizable monomer, a filler, and a polymerization initiator as components, and other component can be appropriately selected and added. According to the purposes, additive components such as water, organic solvents, polymerization inhibitors, pigments and antibacterial agents may be appropriately blended.

As the polymerization initiator used in the present invention, compounds used usually in the dental composition can be used.

The polymerization initiator is generally classified into a chemical polymerization initiator and a photopolymerization initiator. Specifically, as the chemical polymerization initiator, for example, organic peroxides such as benzoyl peroxide, parachlorobenzoyl peroxide, 2,4-dichlorobenzoyl peroxide, acetyl peroxide, lauroyl peroxide, tertiary butyl peroxide, cumen hydroperoxide, 2,5-dimethylhexane-2,5-dihydroperoxide, methyl ethyl ketone peroxide and tertiary butylperoxy benzoate; and azo compounds such as azobisisobutyronitrile, methyl azobisisobutyrate and azobiscyanovaleric acid are preferably used.

The polymerization can also be carried out at a normal temperature by using the organic peroxide in combination with an amine compound. As the amine compound, for example, a secondary or tertiary amine in which an amine group is bonded with an aryl group is preferably used in view of promotion of curing. For example, N,N-dimethyl-p-toluidine, N,N-dimethylaniline, N,N-β-hydroxyethyl-aniline, N,N-di(β-hydroxyethyl)-aniline, N,N-di(β-hydroxyethyl)-p-toluidine, N-methyl-aniline and N-methyl-p-toluidine are preferred.

It is also preferred to use a combination of the organic peroxide and the amine compound in combination with a sulfinate or borate. Examples of sulfinates include sodium benzenesulfinate, lithium benzenesulfonate and sodium p-toluenesulfonate, and examples of the borate include sodium salt, lithium salt, potassium salt, magnesium salt, tetrabutylammonium salt and tetramethylammonium salt of trialkylphenylboron and trialkyl(p-fluorophenyl)boron (an alkyl group is an n-butyl group, n-octyl group, n-dodecyl group, etc.). Organoboron compounds which generate a radical upon reaction with oxygen or water, such as tributylborane and tributylborane partial oxide can also be used as an organic metal type polymerization initiator.

As the photopolymerization initiator, a photosensitizer which generates a radical upon exposure to light can be used. Examples of the photosensitizer to ultraviolet rays include benzoin-based compounds such as benzoin, benzoin methyl ether and benzoin ethyl ether; benzophenone-based compounds such as acetoinbenzophenone, p-chlorobenzophenone and p-methoxybenzophenone; and thioxanthone-based compounds such as thioxanthone, 2-chlorothioxanthone, 2-methylthioxanthone, 2-isopropylthioxanthone, 2-methoxythioxanthone, 2-hydroxythioxanthone, 2,4-diethylthioxanthone and 2,4-diisopropylthioxanthone. A photosensitizer which initiates the polymerization upon exposure to visible light is preferably used since it does not require ultraviolet rays which are harmful to the human body. Examples thereof include a-diketones such as benzil, camphorquinone, a-naphthil, acetonaphcene, p,p′-dimethoxybenzyl, p,p′-dichlorobenzylacetil, pentanedione, 1,2-phenanthrenequinone, 1,4-phenanthrenequinone, 3,4-phenanthrenequinone, 9,10-phenanthrenequinone and naphthoquinone. Preferably, camphorquinone is used.

It is also preferred to use the photosensitizer in combination with photopolymerization promoters. Particularly, when tertiary amines are used as photopolymerization promoters, it is more preferred to use compounds in which a nitrogen atom is directly substituted on an aromatic group. Examples of the photopolymerization promoters include N,N-dimethylaniline, N,N-diethylaniline, N,N-di-n-butylaniline, N,N-dibenzylaniline, N,N-dimethyl-p-toluidine, N,N-dimethyl-m-toluidine, N,N-diethyl-p-toluidine, p-bromo-N,N-dimethylaniline, m-chloro-N,N-dimethylaniline, p-dimethylaminobenzaldehyde, p-dimethylaminoaetophenone, p-dimethylaminobenzoic acid, ethyl p-dimethylaminobenzoate, amino p-dimethylaminobenzoate, methyl N,N-dimethylanthranilate, N,N-dihydroxyethylaniline, N,N-dihydroxyethyl-p-toluidine, p-dimethylaminophenylalcohol, p-dimethylaminostyrene, N,N-dimethyl-3,5-xylidine, 4-dimethylaminopyridine, N,N-dimethyl-a-naphthylamine, N,N-dimethyl-β-naphthylamine, 4-dimethylaminobenzophenone, 4-diethylaminobenzophenone, 4-dipropylaminobenzophenone, 3-dimethylaminobenzophenone, 3-diethylaminobenzophenone, 2-dimethylaminobenzophenone and 2-diethylaminobenzophenone. Examples of the aliphatic tertiary amine include tributylamine, tripropylamine, triethylamine, N-methyldiethanolamine, N-ethyldiethanolamine, N,N-dimethylhexylamine, N,N-dimethyldodecylamine, N,N-dimethylstearylamine, N,N-dimethylaminoethyl methacrylate and N,N-diethylaminoethyl methacrylate. As another photopolymerization promoters, for example, barbituric acids such as 5-butylbarbituric acid and 1-benzyl-5-phenylbarbituric acid; and tin compounds such as dibutyltin diacetate, dibutyltin dimaleate, dioctyltin dimaleate, dioctyltin dilaurate, dibutyltin dilaurate, dioctyltin diversatate, dioctyltin S,S′-bis-isooctylmercaptoacetate and tetramethyl-1,3-diacetoxydistannoxane can be preferably used. At least one kind selected from among these photopolymerization promoters can be used alone, or two or more kinds of them can be used in combination.

In order to improve photopolymerization accelerating ability, it is effective to add, in addition to the tertiary amine, oxycarboxylic acids such as citric acid, malic acid, tartaric acid, glycolic acid, gluconic acid, a-oxyisobutyric acid, 2-hydroxypropanonic acid, 3-hydroxypropanonic acid, 3-hydroxybutanoic acid, 4-hydroxybutanoic acid and dimethylolpropionic acid.

The dental resin-based cement is composed of a plurality of compositions, usually two compositions. In the case of using the dental resin-based cement, the kneading work has conventionally been carried out on a kneading paper by dentists using a spatula. In recent years, a container equipped with mixing elements, namely, an automix container is used in some cases. In the case of a cement using the automix container, since an aspect of the composition is composed of two pastes and the kneading work is automatically carried out in a chip having mixing elements at a container tip, there is no troublesomeness of the kneading work as described above. However, it is necessary to securely carry out kneading in the chip. If sufficient kneading is not carried out, it becomes impossible to obtain high mechanical properties and high bonding properties as features of the dental resin-based cement similar to the above case.

Therefore, when the container equipped with mixing elements, namely, the automix container is used in the paste ejecting site, since sufficient kneading work must be automatically carried out in the chip having mixing elements at a container tip, it is necessary to study about mixability and ejecting properties of both pastes using two pastes in combination.

When the value obtained from a linear approximation curve passing through the origin is expressed as an “estimate”, the linear approximation curve passing through the origin is an “estimation curve” and is represented by the following equation (2):

[Equation 1]

ŷ=βx Equation (2)

wherein y denotes an “estimate”, β denotes a proportionality constant, and χ denotes 20 viscosity values of a first paste as an independent variable.

Herein, the proportionality constant β is obtained from a least square method and is represented by the following equation (3):

$$\begin{matrix}[ Equation 2 ] & \\ β = \frac{{\sum }_{i = 1}^{20} {x}_{i} {y}_{i}}{{\sum }_{i = 1}^{20} {x}_{i}^{2}} & Equation ( 3 )\end{matrix}$$

wherein i denotes an i-th viscosity value, χ denotes 20 viscosity values of a first paste as an independent variable, and denotes 20 viscosity values of a second paste as a dependent variable.

Furthermore, in the case of a straight line passing through the origin, determination coefficient R² is represented by (square-sum of estimate)/(square-sum of y). The determination coefficient represents what proportion of the dependent variable is explainable by the independent variable. Low value means that predictive ability of the resultant “estimation curve” is low (written by Snedecor and Cochran (translated by Hatamura, Okuno and Tsumura), “Tokeiteki-Hoho Gensho 6th ed.”, Iwanami Shoten, Publishers, 1972, chapter 6, Section 18, refer to “Application of Straight Line Passing Origin”).

Herein, regarding the coefficient β and R², when 20 viscosity values are the very same, namely, the same flow behavior is exhibited, both the proportionality constant β and the determination coefficient R² of the linear approximation curve passing through the origin are 1.

Namely, mixability and ejecting properties of both pastes when using two pastes in combination are important elements in a practical operability of the dental resin-based cement composition, and can be evaluated by the above coefficients β and R².

The dental resin-based cement composition in the present invention contains a polymerizable monomer, a filler, and a polymerization initiator as components, and other components can be appropriately selected and added. According to the purposes, additive components such as water, organic solvents, polymerization inhibitors, pigments and antibacterial agents may be appropriately blended.

Since use of the polymer finally decreases the amount of the inorganic filler filled, it is preferred not to blend the polymer. When the polymer is blended, preferred properties depending on the inorganic filler, for example, X-ray opaqueness, fluorine sustained releasability, high elastic modulus and transparency deteriorate.

Since the polymer is not dispersed in a preferred resin system which has conventionally been used, sufficient strength and durability required as the dental material cannot be obtained.

Next, the method for controlling flow properties of a dental resin-based cement composition of the present invention comprises mixing, upon use, at least two kinds of pastes, each comprising (a) 20 to 50 parts by weight of a polymerizable monomer composition, and (b) 50 to 80 parts by weight of a filler consisting of an inorganic compound, or an organic composite comprising the inorganic compound, amounts of (a) and (b) are being based on conversion of 100 parts by weight of the dental resin-based cement composition paste, and the filler containing (c) 2.0 to 10.0 parts by weight of fine silica particles based on the above conversion and using the mixture as the dental resin-based cement composition, wherein as one paste of at least two kinds of pastes, a paste in which (d) a yield viscosity of the pastes is from 100 to 3,000 [Pa·s] and (e) a thixotropy index of the pastes is 3.0 or more, and as another paste, a paste in which (d) a yield viscosity of the pastes is from 70 to 4,000 [Pa·s] and in which (e) the paste has the same thixotropy index are selected and used.

Herein, the terms “polymerizable monomer composition”, “filler and fine silica particles” and “yield viscosity and thixotropy index” are as defined above. According to the method of the present invention, there is provided a method for controlling flow properties of a dental resin-based cement composition, which comprises mixing at least two kinds of pastes, each comprising a polymerizable monomer composition, a filler and fine silica particles in each predetermined amount, and selecting, as one paste of the at least two kinds of pastes, a paste having a yield viscosity of 100 to 3,000 [Pa·s] and a thixotropy index of 3.0 or more and selecting, as another paste, a paste having a yield viscosity of 70 to 4,000 [Pa·s] and a thixotropy index of 3.0 or more and using these pastes.

The method for controlling flow properties of a dental resin-based cement composition of the present invention comprises mixing, upon use, at least two kinds of pastes, each comprising (a) 20 to 50 parts by weight of a polymerizable monomer composition, and (b) 50 to 80 parts by weight of a filler composed of an inorganic compound or an organic composite containing the inorganic compound, amounts of (a) and (b) being based on conversion of 100 parts by weight of the dental resin-based cement composition paste, and the filler containing (c) 2.0 to 10.0 parts by weight of fine silica particles based on the above conversion, and using the mixture as the dental resin-based cement composition, wherein as each of the at least two kinds of pastes, a paste having (d) a yield viscosity of 100 to 3,000 [Pa·s] and (e) a thixotropy index of 3.0 or more is selected and used.

According to the method of the present invention, there is provided a method for controlling flow properties of a dental resin-based cement composition, which comprises mixing at least two kinds of pastes each comprising a polymerizable monomer composition, a filler and fine silica particles in each predetermined amount, and using the mixture, wherein each of the at least two kinds of pastes having a yield viscosity of 100 to 3,000 [Pa·s] and a thixotropy index of 3.0 or more is selected and used, are mixed before used.

As a further aspect of the method for controlling flow properties of the dental resin-based cement composition of the present invention, there is provided a method for controlling flow properties of a dental resin-based cement composition, which comprises selecting, as two kinds of pastes, two kinds of pastes in which (f) is such that letting the paste having the smaller yield viscosity (d) to be a first paste and the other paste to be a second paste, 20 viscosity values are determined by using 20 measurement points within the range from 0.1 [s⁻¹] to 10 [s⁻¹] so as to include shear rates 0.1 [s⁻¹] and 10 [s⁻¹] and to make the intervals between logarithms of the shear rates equal, and when a proportionality constant β and a determination coefficient R² of a linear approximation curve through the origin calculated with respect to a function in which 20 viscosity values of the first paste are independent variables and 20 viscosity values of the second paste are dependent variables, β is within the range from 1.00 to 15.00 and R² is within the range from 0.50 to 1.00, and using these pastes.

### EXAMPLES

The present invention is described in detail below by way of Examples and Comparative Examples. The present invention is not limited to these Examples.

### 1. Preparation of Dental Resin-Based Cement Composition

According to the formulation shown in Table 1, the following components (a), (b) and (c) were mixed using a mortar to prepare a dental resin-based cement composition.

Pastes of Examples 1 to 17 and Comparative Examples 1 to 18 shown in Table 1 were made to contain 0.2 part by weight of camphorquinone as a photopolymerization initiator and 0.2 part by weight of ethyl p-dimethylaminobenzoate as a photopolymerization promoter.

Pastes of Example 11 and Example 13 shown in Table 1 were made to contain 0.3 part by weight of benzoyl peroxide as a chemical polymerization initiator and 0.1 part by weight of 1-benzyl-5-phenylbarbituric acid as a chemical polymerization promoter.

Furthermore, pastes of Examples 1 to 10, 12 and 14 to 17, and Comparative Examples 1 to 18 shown in Table 1 were made to contain 1.5 parts by weight of N,N-dimethyl-p-toluidine as a chemical polymerization promoter.

### (a) Polymerizable Monomer Composition

Using 2,2-bis(4-(2-hydroxy-3-methacryloxypropoxy)phenyl)propane urethane dimethacrylate and triethylene glycol dimethacrylate, a polymerizable monomer composition was prepared according to the formulation shown in Table 1.

### (b) Filler Component

As the filler component other than fine silica particles, amorphous glass fillers having an average particle diameter of 1 μm and/or 3 μm subjected to a surface treatment with γ-methacryloxypropyltrimethoxysilane were used. The amounts of the filler component in Examples or Comparative Examples are shown in Table 1.

### (c) Fine Silica Particles

### Example 1, Example 4, Example 9, and Comparative Examples 1 to 3

AEROSIL R805 manufactured by Degussa (specific surface area by a BET method: 150±25 [m²/g]; pH value in 4% water dispersion (water:methanol=1:1 solution): 3.5 to 5.5; average diameter of primary particles: about 12 nm; apparent specific gravity: about 50; ignition loss at 1,000° C. for 2 hours: 5 to 7 [%]; hydrophobic fine silica particles subjected to surface treatment with octylsilane) was used.

### Example 5, Example 13, Example 17, and Comparative Examples 9, 10, 15 and 18

RY200 manufactured by NIPPON AEROSIL CO., LTD. (specific surface area by a BET method, 100±20 [m²/g]; pH value in 4% water dispersion (water:methanol=1:1 solution): 4 to 7; average diameter of primary particles: about 12 m; apparent specific gravity: about 50; ignition loss at 1,000° C. for 2 hours: 4 to 6 [%]; hydrophobic fine silica particles subjected to surface treatment with dimethylsilicone oil) was used.

### Example 2, 3, 6 to 8, 10 to 12 and 14 to 16, and Comparative Example 5, Comparative Examples 11 to 14 and Comparative Examples 16 to 17

AEROSIL R8200 manufactured by Degussa (specific surface area by a BET method: 160±25 [m²/g]; pH value in 4% water dispersion (water:methanol=1:1 solution): about 5.0; apparent specific gravity: about 140; hydrophobic fine silica particles subjected to surface treatment with hexamethyldisilazane) was used.

### Comparative Example 4

AEROSIL R711 manufactured by Degussa (specific surface area by a BET method: 150±25 [m²/g]; pH value in 4% water dispersion (water:methanol=1:1 solution): 4 to 6; average diameter of primary particles: about 12 m; apparent specific gravity: about 60; ignition loss at 1,000° C. for 2 hours: 6 to 11 [%]; hydrophobic fine silica particles subjected to surface treatment with methacrylsilane) was used.

### Comparative Examples 6 to 8

R972V manufactured by NIPPON AEROSIL CO., LTD. (specific surface area by a BET method, 110±20 [m²/g]; pH value in 4% water dispersion (water:methanol=1:1 solution): 4.0 to 5.5; average diameter of primary particles: about 16 nm; apparent specific gravity: about 90; ignition loss at 1,000° C. for 2 hours: 2 [%] or less; hydrophobic fine silica particles subjected to surface treatment with methyl group) was used.

The amounts of the filler component in Examples or Comparative Examples are shown in Table 1.

### 2. Method for Measurement of Yield Viscosity and Thixotropy Index of Paste

STRESSTECH Rheometer manufactured by Rheologica Instruments was used.

The measurement conditions are show below.

1) A cone-flat plate jig in which an angle of a sample interval is 40 is used.
2) Measurement is carried out under an environment at a temperature of 23° C. under atmospheric pressure.
3) Steady flow viscosity at a shear rate of 0.1 to 10 [s⁻¹] is measured.
4) Measurement interval is determined by using 20 measurement points within the range from 0.1 [s⁻¹] to 10 [s⁻¹] so as to include shear rates 0.1 [s⁻¹] and 10 [s⁻¹] and to make the intervals between logarithms of the shear rates equal.
5) In order to make stress history of a measurement sample constant, measurement is initiated 10 seconds after applying a shear rate of 10 [s⁻¹] for 60 seconds.

Herein, a steady flow viscosity at a shear rate of 0.1 [s⁻¹] is taken as a yield viscosity of the paste.

The thixotropy index is given by Equation (1) where ρ_{0.1} [Pa s] denotes a viscosity at a shear rate of 0.1 [s⁻¹] and ρ₁₀ [Pa·s] denotes a viscosity at a shear rate of 10 [s⁻¹].

Thixotropy index=ρ_{0.1}/ρ₁₀ Equation (1)

A yield viscosity and a thixotropy index are shown in Table 1.

TABLE 1
Preparation Table of Components of Dental Resin-Based Cement Composition,
and Yield Viscosity, Thixotropy Index and Results of Questionary Survey 1

| | | | | | | Filler component [Parts by | | | |
|---|---|---|---|---|---|---|---|---|---|
| | | Polymerizable monomer | | | | weight] | | | |
| | Examples | composition (a) [Parts by | | | | | | (c) | |
| | and | weight] | | | | | | Fine | |
| | Comparative | Bis- | | | | | | silica | |
| No. | Examples | GMA | UDMA | TEGMA | Total | 1 μm | 3 μm | particles | Total |
| 1 | Example 1 | 4.0 | | 16.0 | 20.0 | | 76.1 | 2.0 | 78.1 |
| 2 | Example 2 | | 12.0 | 8.0 | 20.0 | | 76.1 | 2.0 | 78.1 |
| 3 | Comparative | | 12.0 | 8.0 | 20.0 | | 77.1 | 1.0 | 78.1 |
| | Example 1 | | | | | | | | |
| 4 | Comparative | | 10.7 | 9.3 | 20.0 | | 76.1 | 2.0 | 78.1 |
| | Example 2 | | | | | | | | |
| 5 | Comparative | 4.0 | | 16.0 | 20.0 | | 77.1 | 1.0 | 78.1 |
| | Example 3 | | | | | | | | |
| 6 | Comparative | 4.0 | | 16.0 | 20.0 | | 72.1 | 6.0 | 78.1 |
| | Example 4 | | | | | | | | |
| 7 | Comparative | 8.0 | | 12.0 | 20.0 | | 77.1 | 1.0 | 78.1 |
| | Example 5 | | | | | | | | |
| 8 | Example 3 | | 14.0 | 9.3 | 23.3 | 4.0 | 67.8 | 3.0 | 74.8 |
| 9 | Example 4 | | 14.1 | 9.4 | 23.5 | | 70.6 | 4.0 | 74.6 |
| 10 | Example 5 | | 15.6 | 10.4 | 26.0 | 9.6 | 59.6 | 2.9 | 72.1 |
| 11 | Example 6 | | 15.6 | 10.4 | 26.0 | 8.0 | 60.5 | 3.6 | 72.1 |
| 12 | Comparative | | 15.9 | 10.6 | 26.5 | 61.1 | | 10.5 | 71.6 |
| | Example 6 | | | | | | | | |
| 13 | Comparative | | 16.6 | 11.1 | 27.7 | 63.9 | | 6.5 | 70.4 |
| | Example 7 | | | | | | | | |
| 14 | Comparative | | 17.3 | 11.5 | 28.8 | 66.7 | | 2.6 | 69.3 |
| | Example 8 | | | | | | | | |
| 15 | Example 7 | 5.3 | 10.0 | 14.6 | 29.9 | 13.3 | 50.7 | 4.2 | 68.2 |
| 16 | Example 8 | | 18.0 | 12.0 | 30.0 | 13.3 | 50.1 | 4.7 | 68.1 |
| 17 | Example 9 | | 19.3 | 12.9 | 32.2 | | 62.2 | 3.8 | 65.9 |
| 18 | Example 10 | | 19.6 | 13.1 | 32.7 | 8.8 | 54.0 | 2.6 | 65.4 |
| 19 | Comparative | | 19.8 | 13.2 | 33.0 | 60.0 | | 5.1 | 65.1 |
| | Example 9 | | | | | | | | |
| 20 | Example 11 | 19.8 | | 20.2 | 40.0 | 52.8 | | 5.3 | 58.1 |
| 21 | Example 12 | 22.0 | | 18.0 | 40.0 | 56.7 | | 2.5 | 59.2 |
| 22 | Comparative | 16.0 | | 23.6 | 39.6 | 48.5 | | 10.0 | 58.5 |
| | Example 10 | | | | | | | | |
| 23 | Example 13 | 18.2 | | 21.4 | 39.6 | 51.2 | | 7.3 | 58.5 |
| 24 | Comparative | 22.5 | | 17.1 | 39.6 | 57.2 | | 1.3 | 58.5 |
| | Example 11 | | | | | | | | |
| 25 | Comparative | 23.0 | | 16.6 | 39.6 | 57.7 | | 0.8 | 58.5 |
| | Example 12 | | | | | | | | |
| 26 | Comparative | | 24.5 | 16.6 | 41.1 | 48.7 | | 8.3 | 57.0 |
| | Example 13 | | | | | | | | |
| 27 | Example 14 | 19.1 | | 28.3 | 47.4 | 41.9 | | 8.8 | 50.7 |
| 28 | Example 15 | | 29.1 | 19.6 | 48.7 | 43.3 | | 7.2 | 50.5 |
| 29 | Example 16 | | 28.1 | 20.0 | 48.1 | 40.0 | | 10.0 | 50.0 |
| 30 | Example 17 | 28.1 | | 20.0 | 48.1 | 45.0 | | 5.0 | 50.0 |
| 31 | Example 18 | 28.1 | | 20.0 | 48.1 | 47.5 | | 2.5 | 50.0 |
| 32 | Example 19 | | 28.1 | 20.0 | 48.1 | 46.2 | | 3.8 | 50.0 |
| 33 | Comparative | 28.1 | | 20.0 | 48.1 | 40.0 | | 10.0 | 50.0 |
| | Example 14 | | | | | | | | |
| 34 | Comparative | 28.1 | | 20.0 | 48.1 | 49.2 | | 0.8 | 50.0 |
| | Example 15 | | | | | | | | |
| 35 | Comparative | | 28.1 | 20.0 | 48.1 | 38.5 | | 11.5 | 50.0 |
| | Example 16 | | | | | | | | |

| | (d) | (e) | Results of questionary survey 1 | | | | |
|---|---|---|---|---|---|---|---|
| | Yield strength | Thixotropy | | Kneading | | | |
| No. | [Pa · s] | index | Sag | resistance | Admixing | Effects | Judgment |
| 1 | 408.0 | 6.4 | 1 | 1 | −1 | 1 | Good |
| 2 | 1390.0 | 12.4 | 2 | −1 | 1 | 2 | Good |
| 3 | 95.4 | 1.4 | −2 | 1 | 1 | 0 | Poor |
| 4 | 65.5 | 1.5 | −2 | −1 | −1 | −4 | Poor |
| 5 | 42.6 | 1.4 | −2 | −1 | 1 | −2 | Poor |
| 6 | 21.6 | 1.0 | −2 | −1 | 1 | −2 | Poor |
| 7 | 336.9 | 2.1 | −1 | −2 | −2 | −5 | Poor |
| 8 | 587.8 | 11.5 | 1 | 2 | 2 | 5 | Good |
| 9 | 2680.0 | 19.0 | 2 | 1 | −1 | 2 | Good |
| 10 | 2119.0 | 18.3 | 2 | 1 | −1 | 2 | Good |
| 11 | 777.5 | 19.2 | 2 | 1 | 1 | 4 | Good |
| 12 | 89.5 | 1.6 | −2 | −2 | −2 | −6 | Poor |
| 13 | 67.0 | 1.9 | −2 | −1 | 1 | −2 | Poor |
| 14 | 30.0 | 1.2 | −2 | −1 | 1 | −2 | Poor |
| 15 | 842.2 | 24.0 | 2 | 2 | 2 | 6 | Good |
| 16 | 468.3 | 30.4 | 2 | 2 | 2 | 6 | Good |
| 17 | 1102.0 | 22.0 | 2 | 2 | 2 | 6 | Good |
| 18 | 73.2 | 3.9 | −1 | 1 | −1 | −1 | Poor |
| 19 | 4262.0 | 23.9 | 2 | −2 | −2 | −2 | Poor |
| 20 | 2784.0 | 27.4 | 2 | 1 | 1 | 4 | Good |
| 21 | 135.3 | 4.5 | 2 | 2 | 2 | 6 | Good |
| 22 | 5924.0 | 30.5 | 2 | −2 | −1 | −1 | Poor |
| 23 | 3570.0 | 33.1 | 2 | −1 | −1 | 0 | Poor |
| 24 | 24.7 | 0.9 | −2 | 1 | 2 | 1 | Poor |
| 25 | 14.0 | 0.8 | −2 | 1 | 2 | 1 | Poor |
| 26 | 4144.0 | 30.3 | 2 | −2 | −2 | −2 | Poor |
| 27 | 1818.0 | 47.6 | 2 | 1 | 1 | 4 | Good |
| 28 | 1510.0 | 32.5 | 2 | 1 | −1 | 2 | Good |
| 29 | 1595.0 | 56.2 | 2 | 2 | −1 | 3 | Good |
| 30 | 884.8 | 26.1 | 2 | 2 | 2 | 6 | Good |
| 31 | 135.0 | 8.6 | 2 | 2 | 2 | 6 | Good |
| 32 | 110.5 | 12.8 | 1 | 2 | 2 | 5 | Good |
| 33 | 6554.0 | 42.0 | 2 | −1 | −1 | 0 | Poor |
| 34 | 7.2 | 1.4 | −2 | 2 | 2 | 2 | Poor |
| 35 | 5200.0 | 28.2 | 2 | −2 | −2 | −2 | Poor |

### 3. Inspection 1 of Operability

First, with respect to operability properties to be estimated in a clinical practice, “sagging” “kneading resistance” and “kneading” were selected and a survey 1 was carried out by dentists, followed by inspection. Meanings of the properties are shown below.

“Sagging” was judged from whether or not the kneaded cement paste sags from a spatula or not after the kneaded cement paste was applied to a restoration or filled in a restoration using the spatula.

“Kneading resistance” means resistance of a cement paste during kneading, although a dental cement must be strongly-kneaded on a cement kneading board using a spatula and this kneading operation is called kneading. The kneading resistance was judged from whether or not it is small.

The term “admixing” means operability when slightly mixing at an initial stage of kneading in case where a dental cement provided as a semifinished product is kneaded to form a finished product, and can be easily judged from the fluidity when evaluation is carried out using a single paste.

The survey 1 was carried out under an environment at 23° C.

The dental resin-based cement is generally provided as a plurality of pastes. However, it is easy to estimate from evaluation of operability of individual paste, as evaluation of operability of the dental resin-based cement, even if the final form is a plurality of pastes.

Therefore, in the inspection 1 of various operabilities, operability of individual paste was evaluated.

In the survey 1, as shown in Table 2, four-grade evaluation was carried out with respect to the above examination items. Evaluation results were scored and the effect was determined as the sum of the obtained scores in each examination item. Scores are shown in Table 1.

TABLE 2
Evaluation Measure of Questionnaire and Corresponding Scores

| Evaluation measure | Scores |
|---|---|
| Extremely excellent | +2 |
| Excellent | +1 |
| Slightly unsatisfactory | −1 |
| Unsatisfactory | −2 |

Criteria for evaluation measure of the survey 1 are shown in Table 3.

TABLE 3
Criteria of Evaluation Measure of Survey 1

| | Measure | | | |
|---|---|---|---|---|
| Examination | | | Slightly | |
| Item | Extremely excellent | Excellent | unsatisfactory | Unsatisfactory |
| Sag | No paste sags from a | No paste sags | A paste tends to | A paste sags from |
| | spatula during an | without slightly | sag slightly from a | a spatula during |
| | operation such as | retaining a shape | spatula during an | an operation such |
| | application to or | during an operation | operation such as | as application to |
| | filling in a | such as application | application to or | or filling in a |
| | restoration and | to or filling in a | filling in a | restoration and |
| | retains a shape. | restoration and | restoration and | exhibits strong |
| | The paste exhibits | retains a shape. | retains a shape. | stringiness. The |
| | no stringiness when | The paste exhibits | The paste exhibits | paste, is not |
| | separated from the | slight stringiness | slight stringiness | suited for a |
| | spatula | when separated from | when separated from | clinical use. |
| | | the spatula. | the spatula. | |
| Kneading | Users scarcely feel | Users slightly feel | Users slightly feel | Users strongly |
| resistance | a resistance at the | a resistance at the | a resistance at the | feel a resistance |
| | beginning of | beginning of | beginning of | at the beginning |
| | kneading and during | kneading but feel | kneading and during | of kneading and |
| | kneading. | no difficulty, and | kneading, but it is | during kneading, |
| | | kneading allows | possible to put | and it is not |
| | | users to scarcely | into a clinical | suited for a |
| | | feel a resistance | use. A decrease in | clinical use. A |
| | | | a resistance by | decrease in a |
| | | | kneading is | resistance by |
| | | | recognized | kneading is not |
| | | | | recognized. |
| Kneading | A paste is | A paste is | A paste exhibits | A paste exhibits |
| | sufficiently mixed | sufficiently mixed | slight viscous | considerable |
| | by kneading. The | by kneading. The | fluidity to a | viscous fluidity |
| | paste exhibits | paste exhibits | kneading operation | to a kneading |
| | smooth fluidity to a | smooth fluidity to | and also exhibits | operation and |
| | kneading operation, | a kneading | stringiness, but | also exhibits |
| | but is not viscous | operation, but is | exhibits sufficient | strong |
| | even when strongly | slightly viscous | mixability by | stringiness, and |
| | kneaded. | when strongly | kneading and can be | it is not easy to |
| | | kneaded. | applied for a | sufficiently |
| | | | clinical use. | knead and it is |
| | | | | not suited for a |
| | | | | clinical use. |

In the survey 1, the case where the sum of obtained scores in each examination item is 1 or more was rated “Good”, whereas the case where the sum is 0 or less was rated “Poor”. The case where the evaluation measure was “unsatisfactory” with respect to one or more items was rated “Poor” as inappropriate paste properties, regardless of the sum of the obtained scores.

### Evaluation 1

In test Nos. 1 to 7, the dental resin-based cement compositions contains 20 parts by weight of the polymerizable monomer composition component (a) and 80 parts by weight of the filler component (b).

In Example 1 and Example 2, 2.0 parts by weight of the fine silica particle component (c) is added so that (d) the yield viscosity is within the range from 100 to 3,000 [Pa·s]. Furthermore, in the paste composition, selection is carried out so that (e) the thixotropy index is 3.0 or more.

In contrast, in Comparative Examples 2 and 4, although the fine silica particle component (c) selected so that (e) the thixotropy index is outside the range of 3.0 or more is added within the range from 2.0 to 10.0 parts by weight, (d) the yield viscosity is adjusted to be outside the range from 100 to 3,000 [Pa·s].

In the dental resin-based cement composition of Comparative Example 5, the fine silica particle component (c) selected so that (e) the thixotropy index is outside the range of 3.0 or more is added within the range from 2.0 to 10.0 parts by weight, and (d) the yield viscosity is adjusted within the range from 100 to 3,000 [Pa·s].

In the dental resin-based cement compositions of Comparative Examples 1 and 3, the fine silica particle component (c) selected so that (e) the thixotropy index is outside the range of 3.0 or more is added outside the range from 2.0 to 10.0 parts by weight, and (d) the yield viscosity is adjusted to be outside the range from 100 to 3,000 [Pa·s].

As is apparent from Table 1, as a result of the survey 1, Examples 1 and 2 satisfying the constituent features (a) to (e) exhibited good operability.

However, regarding operability of Comparative Examples 1 to 5 which do not simultaneously satisfy the constituent features (a) to (e), “sag” and “flow resistance” characteristics were particularly poor and operability was poor.

The dental resin-based cement compositions of test Nos. 8 to 16 contains 23.3 to 30.0 parts by weight of the polymerizable monomer composition component (a) and 76.7 to 70.0 parts by weight of the filler component (b).

In Examples 3 to 8, the fine silica particle component (c) in which (e) the thixotropy index is 3.0 or more is added within the range from 2.0 to 10.0 parts by weight, and (d) the yield viscosity is adjusted within the range from 100 to 3,000 [Pa·s].

In the dental resin-based cement composition of Comparative Example 6, the fine silica particle component (c) in which (e) the thixotropy index is outside the range of 3.0 or more is added within the range from 2.0 to 10.0 parts by weight, and (d) the yield viscosity is adjusted to be outside the range from 100 to 3,000 [Pa·s].

In the dental resin-based cement compositions of Comparative Examples 7 and 8, the fine silica particle component (c) in which (e) the thixotropy index is outside the range of 3.0 or more is added within the range from 2.0 to 10.0 parts by weight, and (d) the yield viscosity is adjusted to be outside the range from 100 to 3,000 [Pa·s].

As is apparent from Table 1, as a result of the survey 1, Examples 3 to 8 satisfying the constituent features (a) to (e) exhibited good operability.

With respect to operability of Examples 7 and 8, highest evaluation was given to all questionary items and operability was particularly excellent.

However, Comparative Examples 6 to 8 which do not simultaneously satisfy the constituent features (a) to (e) exhibited poor operability.

Like Comparative Example 6, when the fine silica particle component (cc) is added outside the range from 2.0 to 10.0 parts by weight, lowest evaluation was given to all questionary items and operability was considerably poor.

The dental resin-based cement compositions of test Nos. 17 to 25 contains 32.2 to 40.0 parts by weight of the polymerizable monomer composition component (a) and 67.8 to 60.0 parts by weight of the filler component (b).

In Examples 9, 11 and 12, the fine silica particle component (c) selected so that (e) the thixotropy index is within the range of 3.0 or more is added within the range from 2.0 to 10.0 parts by weight, (d) the yield viscosity is adjusted within the range from 100 to 3,000 [Pa·s].

In contrast, in the dental resin-based cement compositions of Comparative Examples 9 and 10, the fine silica particle component (c) in which (e) the thixotropy index is 3.0 or more is added within the range from 2.0 to 10.0 parts by weight, and (d) the yield viscosity is adjusted to be outside the range from 100 to 3,000 [Pa·s].

In the dental resin-based cement compositions of Comparative Examples 11 and 12, the fine silica particle component in which (e) the thixotropy index is outside the range of 3.0 or more is added outside the range from 2.0 to 10.0 parts by weight, and (d) the yield viscosity is adjusted to be outside the range from 100 to 3,000 [Pa·s].

As is apparent from Table 1, as a result of the survey 1, Examples 9, 11 and 12 satisfying the constituent features (a) to (e) exhibited good operability.

With respect to operability of Examples 9 and 12, highest evaluation was given to all questionary items and operability was particularly excellent.

In Example 11, the fine silica particle component (cc) is added in a comparatively large amount when compared with Examples 9 and 11 and (d) the yield viscosity is comparatively high. Therefore, “kneading resistance” and “admixing” characteristics of the items of the survey 1 were inferior when compared with Examples 9 and 12.

However, Comparative Examples 9 to 12 which do not simultaneously satisfy the constituent features (a) to (e) exhibited poor operability.

In Comparative Examples 11 to 12 in which the amount of the fine silica particle component (cc) added is less than 2.0 parts by weight, the fine silica particles (c) in which (e) the thixotropy index is within the range from 0.9 to 0.8 are selected. In the results of the survey 1, “kneading resistance” and “admixing” characteristics were good, while “sag” characteristics were considerably poor.

The dental resin-based cement compositions of test Nos. 26 to 35 contain 41.5 to 50.0 parts by weight of the polymerizable monomer composition component (a) and 58.5 to 50.0 parts by weight of the filler component (b).

In Examples 14 to 19, the fine silica particle component (c) selected so that (e) the thixotropy index is 3.0 or more is added in the range from 2.0 to 10.0 parts by weight, and (d) the yield viscosity is adjusted within the range from 100 to 3,000 [Pa·s].

In contrast, in the dental resin-based cement compositions of Comparative Examples 13 to 14, although the fine silica particle component (c) in which (e) the thixotropy index is 3.0 or more is added within the range from 2.0 to 10.0 parts by weight, (d) the yield viscosity is adjusted to be outside the range from 100 to 3,000 [Pa·s].

In the dental resin-based cement composition of Comparative Example 15, (d) the yield viscosity is adjusted to be outside the range from 100 to 3,000 [Pa·s] by the amount of the fine silica particle component (c), in which (e) the thixotropy index is outside the range of 3.0 or more, is outside the range from 2.0 to 10.0 parts by weight.

In the dental resin-based cement composition of Comparative Example 16, (d) the yield viscosity is adjusted to be outside the range from 100 to 3,000 [Pa·s] by the amount of the fine silica particle component (c), in which (e) the thixotropy index is 3.0 or more, is outside the range from 2.0 to 10.0 parts by weight.

As is apparent from Table 1, as a result of the survey 1, Examples 14 to 19 satisfying the constituent features (a) to (e) exhibited good operability.

With respect to operability of Examples 17 and 18, highest evaluation was given to all questionary items and operability was particularly excellent.

However, Comparative Examples 13 to 16 which do not simultaneously satisfy the constituent features (a) to (e) exhibited poor operability.

Like Comparative Examples 13 to 14 and 16, when the content of fine silica particles is comparatively high such as 8.3 to 11.5 parts by weight, deterioration of “kneading resistance” and “admixing” characteristics is caused by excess increase in yield viscosity, and operability is poor.

In contrast, like Comparative Examples 15, when the content of fine silica particles is low such as 0.8 part by weight, both yield viscosity and thixotropy index are outside the range, and particularly deterioration of “sag” characteristics is recognized and operability is poor.

### 4. Inspection 2 of Operability

First, with respect to operability properties to be estimated in a clinical practice, “sagging” “kneading resistance” and “kneading” were selected and survey 2 was carried out by dentists, followed by inspection. The meanings of the properties are shown below.

“Sagging” was judged from whether or not the kneaded cement paste sags from a spatula or not after the kneaded cement paste was applied to a restoration or filled in a restoration using the spatula.

“Kneading resistance” means resistance of a cement paste during kneading, although a dental cement must be strongly kneaded on a cement kneading board using a spatula and this kneading operation is called kneading. The kneading resistance was judged from whether or not it is small.

The term “admixing” means operability when slightly mixing at an initial stage of kneading in case where a dental cement provided as a semifinished product is kneaded to form a finished product, and can be easily judged from the fluidity when evaluation is carried out using a single paste.

Therefore, in the evaluation of operability of the dental resin-base cement in the inspection 2 of various operabilities, two component dental resin-based cements composed of two kinds of pastes were prepared and the combinations are shown in Table 6 and Table 7.

The survey 2 was carried out under an environment at 23° C.

In the survey 2, as shown in Table 4, four-grade evaluation was carried out with respect to the above examination items. Evaluation results were scored and the effect was determined as the sum of the obtained scores in each examination item. Scores are shown in Table 6 and Table 7.

TABLE 4
Evaluation Measure of Questionnaire and Corresponding Scores

| Evaluation measure | Scores |
|---|---|
| Extremely excellent | +2 |
| Excellent | +1 |
| Slightly unsatisfactory | −1 |
| Unsatisfactory | −2 |

Criteria for evaluation measure of the survey 2 are shown in Table 5.

TABLE 5
Criteria of Evaluation Measure of Survey 2

| | Measure | | | |
|---|---|---|---|---|
| Examination | | | Slightly | |
| Item | Extremely excellent | Excellent | unsatisfactory | Unsatisfactory |
| Sag | No paste sags from a | No paste sags | A paste tends to | A paste sags from |
| | spatula during an | without slightly | sag slightly from a | a spatula during |
| | operation such as | retaining a shape | spatula during an | an operation such |
| | application to or | during an operation | operation such as | as application to |
| | filling in a | such as application | application to or | or filling in a |
| | restoration and | to or filling in a | filling in a | restoration and |
| | retains a shape. The | restoration and | restoration and | exhibits strong |
| | paste exhibits no | retains a shape. | retains a shape. | stringiness. The |
| | stringiness when | The paste exhibits | The paste exhibits | paste is not |
| | separated from the | slight stringiness | slight stringiness | suited for a |
| | spatula | when separated from | when separated from | clinical use. |
| | | the spatula. | the spatula. | |
| Kneading | Users scarcely feel | Users slightly feel | Users slightly feel | Users strongly |
| resistance | a resistance at the | a resistance at the | a resistance at the | feel a resistance |
| | beginning of | beginning of | beginning of | at the beginning |
| | kneading and during | kneading but feel | kneading and during | of kneading and |
| | kneading. | no difficulty, and | kneading, but it is | during kneading, |
| | | kneading allows | possible to put | and it is not |
| | | users to scarcely | into a clinical | suited for a |
| | | feel a resistance | use. A decrease in | clinical use. A |
| | | | a resistance by | decrease in a |
| | | | kneading is | resistance by |
| | | | recognized | kneading is not |
| | | | | recognized. |
| Kneading | A paste is | A paste is | A paste exhibits | A paste exhibits |
| | sufficiently mixed | sufficiently mixed | slight viscous | considerable |
| | by kneading. The | by kneading. The | fluidity to a | viscous fluidity |
| | paste exhibits | paste exhibits | kneading operation | to a kneading |
| | smooth fluidity to a | smooth fluidity to | and also exhibits | operation and also |
| | kneading operation, | a kneading | stringiness, but | exhibits strong |
| | but is not viscous | operation, but is | exhibits sufficient | stringiness, and |
| | even when strongly | slightly viscous | mixability by | it is not easy to |
| | kneaded. | when strongly | kneading and can be | sufficiently knead |
| | Furthermore, | kneaded. | applied for a | and it is not |
| | mixability of two or | Furthermore, | clinical use. | suited for a |
| | more kinds of pastes | mixability of two | Furthermore, | clinical use. |
| | is excellent. | or more kinds of | mixability of two | Furthermore, |
| | | pastes is | or more kinds of | mixability of two |
| | | excellent. | pastes is | or more kinds of |
| | | | excellent. | pastes is |
| | | | | excellent. |

In the survey 2, the case where the sum of obtained scores in each examination item is 1 or more was rated “Good”, whereas the case where the sum is 0 or less was rated “Poor”. The case where the evaluation measure was “unsatisfactory” with respect to one or more items was rated “Poor” as inappropriate paste properties, regardless of the sum of the obtained scores.

Furthermore, when a container equipped with mixing elements at the paste ejecting site, namely, an automix container is used, since sufficient kneading operation must be automatically carried out in a chip having mixing elements at a container tip, mixability and ejecting properties of both pastes were evaluated in the survey 3 using combinations of pastes shown in Table 6 and Table 7.

Herein, the coefficient β and R² in Table 6 and Table 7 are a proportionality constant β and a determination coefficient R² regarding two kinds of pastes. When the paste having a small yield viscosity (d) is a first paste and the other paste is a second paste. 20 viscosity values are determined by using 20 measurement points within the range from 0.1 [s⁻¹] to 10 [s⁻¹] so as to include shear rates 0.1 [s⁻¹] and 10 [s⁻¹] and to make the intervals between logarithms of the shear rates equal. A proportionality constant β and a determination coefficient R² of a linear approximation curve passing through the origin are calculated with respect to a function in which 20 viscosity values of the first paste are dependent variables and 20 viscosity values of the second paste are independent variables. In such a measuring method, the case where the proportionality constant β is within the range from 1.00 to 15.00 and the determination coefficient R² is within the range from 0.50 to 1.00 is the constituent feature (f).

Herein, when 20 viscosity data of the first paste and the second paste are entirely the same, namely, the first paste and the second paste exhibit the same flow behavior, the proportionality constant β and determination coefficient R² of the linear approximation curve passing through the origin simultaneously represent 1.

In the survey 3, when one or more items are rated “Poor”, paste properties were judged as inappropriate paste properties.

The survey 3 was carried out under an environment at 23° C.

### Evaluation 2

Test Nos. 36 to 53 describe the results of the test in which the survey 2 and survey 3 were carried out as the evaluation of operability of two-component dental resin-based cement compositions using the pastes of Example 12 and Comparative Examples 1 to 16 in combination.

All results of the survey 2 with respect to Comparative Examples 17 and 21 to 22 were good and the dental resin-based cement composition had good operability. However, regarding the survey 3, mixability and/or ejecting properties were poor and the dental resin-based cement composition exhibited inappropriate operability. Therefore, the two-component dental resin-based cement composition exhibited good operability as a hand kneading type supply system which is a conventional method. It is possible to judge the two-component dental resin-based cement composition to be inappropriate since an automix container as a comparatively new supply system cannot be used.

In Comparative Examples 17 and 21 to 22, R² is at most 0.43 of Comparative Example 17, which shows that flow properties of the second paste is quite different from flow properties of the first paste and makes the evaluation of the case of using the automix container worse.

It cannot be said that Comparative Examples 17 and 21 to 22 have good operability. In this case, these comparative examples satisfy the constituent features (a) to (e), but do not satisfy the constituent feature (f).

Test Nos. 45, 46 and 48 to 53 describe the results of the test in which the survey 2 and survey 3 were carried out as the evaluation of operability of two-component dental resin-based cement compositions using the pastes of Example 12 and Comparative Examples 9, 10 and 11 to 16 in combination.

All judgments of the results of the survey 2 with respect to Comparative Examples 25, 26 and 27 to 32 were inappropriate and operability of the dental resin-based cement composition was poor. Regarding the survey 3, mixability and ejecting properties were poor and operability of the dental resin-based cement composition was inappropriate. Therefore, it is possible to judge that the two-component dental resin-based cement composition is inappropriate since it cannot be said that the two-component dental resin-based cement composition have no good operability to a hand kneading type supply system as a conventional method and an automix container as a comparatively new supply system has not good operability.

Regarding Comparative Examples 25, 26 and 27 to 32 in which all surveys were rated poor, operability of the dental resin-based cement composition in which β is 15.00 or more and/or R² is outside the range from 0.50 to 1.00 was quite inappropriate. Therefore, it is necessary that individual paste of the two-component dental resin-based cement composition satisfies the constituent features (a) to (e) and (f).

Test Nos. 54 to 70 describe the results of the test in which the survey 2 and survey 3 were carried out as the evaluation of operability of two-component dental resin-based cement compositions using the pastes of Example 12 and Comparative Examples 1 to 9, 11, 12 and 14 to 19 in combination.

All results of the survey 2 with respect to Examples 22 to 38 were good and operability of the dental resin-based cement composition was good. Regarding the survey 3, mixability and ejecting properties were appropriate and operability of the dental resin-based cement composition was appropriate. Therefore, the two-component dental resin-based cement composition has good operability as a hand kneading type supply system which is a conventional method and an automix container as a comparatively new supply system can be used with good operability.

Therefore, it can be said that operability of a hand kneading type supply system as a conventional method is good and the two-component dental resin-based cement composition, which can also use an automix container as a comparatively new supply system with good operability, must satisfy the constituent features (a) to (e) and also satisfies the constituent feature (f).

Test Nos. 71, 75 and 76 describe the results of the test in which the survey 2 and survey 3 were carried out as the evaluation of operability of two-component dental resin-based cement compositions using the pastes of Example 15 and Comparative Examples in combination.

All results of the survey 2 with respect to Comparative Examples 33, 37 and 38 and operability as the dental resin-based cement composition was good. However, regarding the survey 3, mixability and/or ejecting properties were poor and operability as the dental resin-based cement composition was inappropriate. Therefore, it can be judged that the two-component dental resin-based cement composition has good operability as a hand kneading type supply system which is a conventional method, but is inappropriate since an automix container as a comparatively new supply system cannot be used.

As is apparent from the results of the surveys 2 and 3 in the two-component dental resin-based cement composition with respect to Comparative Examples 33, 37 and 38, operability as a hand kneading type supply system which is a conventional method did not have a general correlation with operability when using an automix container as a comparatively new supply system and that indicators different from operability evaluation as a hand kneading type supply system such as β and R² must be required.

Therefore, it can be judged that operability in Comparative Examples 33, 37 and 38 in which both first and second pastes of the dental resin-based cement composition satisfy the constituent features (a) to (e) and do not satisfy the constituent feature (f) is inappropriate.

Test Nos. 72 to 74, 77 to 78, 80 to 81 and 83 to 88 describe the results of the test in which the survey 2 and survey 3 were carried out as the evaluation of operability of two-component dental resin-based cement compositions using the pastes of Example 15 and Comparative Examples in combination.

All judgments of the results of the survey 2 with respect to Comparative Examples 34 to 36, 39 to 40, 41 to 42 and 43 to 48 were poor and operability as the dental resin-based cement composition was inappropriate. Regarding the survey 3, mixability and/or ejecting properties were poor and operability as the dental resin-based cement composition was inappropriate. Therefore, it is possible to judge that the two-component dental resin-based cement composition is inappropriate since it cannot be said that the two-component dental resin-based cement composition have no good operability to a hand kneading type supply system as a conventional method and an automix container as a comparatively new supply system has not good operability.

Either the first or second paste of the dental resin-based cement composition in Comparative Examples 34 to 36, 39 to 40, 41 to 42 and 43 to 48 does not satisfy the constituent features (a) to (e).

Test Nos. 89 to 105 describe the results of the test in which the survey 2 and survey 3 were carried out as the evaluation of operability of two-component dental resin-based cement compositions using the pastes of Examples 1 to 9, 11, 12 and 14 to 19 in combination.

Since the combination of pastes of two-component resin-based cement composition in Examples 52 to 57 is a dental resin-based cement composition whose operability of “sag”, “kneading resistance” and “admixing” was judged good in the evaluation 1, all results of the survey 2 was good.

Furthermore, the results of the survey 3 in Examples 52 to 57 are judged to be good and an automix container as a new supply system can be judged to have good operability.

Therefore, in order to have good operability, it is necessary that individual paste of two kinds of pastes satisfies the constituent features (a) to (e) and also satisfies the constituent feature (f) in the combination of pastes.

TABLE 6
Preparation Table of Two-Components Dental Resin-Based Cement Composition, and β
value, R² value and Results of Questionary Surveys 2, 3

| | Examples and | Combination of pastes | | Results of questionary survey 2 | | | | | | | Results of questionary survey 3 | | |
|---|---|---|---|---|---|---|---|---|---|---|---|---|---|
| Test | Comparative | | Second | | Kneading | | | Judg- | | | | Ejection | |
| No. | Examples | First paste | paste | Sag | resistance | Admixing | Effects | ment | β | R² | Mixability | properties | Judgment |
| 36 | Comparative | Comparative | Example 12 | 2 | 1 | 1 | 4 | Good | 1.09 | 0.43 | Poor | Poor | Poor |
| | Example 17 | Example 1 | | | | | | | | | | | |
| 37 | Comparative | Comparative | Example 12 | −1 | 1 | −1 | −1 | Poor | 1.69 | 0.16 | Poor | Poor | Poor |
| | Example 18 | Example 2 | | | | | | | | | | | |
| 38 | Comparative | Comparative | Example 12 | −1 | 1 | −1 | −1 | Poor | 2.49 | 0.45 | Poor | Poor | Poor |
| | Example 19 | Example 3 | | | | | | | | | | | |
| 39 | Comparative | Comparative | Example 12 | −2 | −1 | 0 | −3 | Poor | 3.68 | 0.12 | Poor | Poor | Poor |
| | Example 20 | Example 4 | | | | | | | | | | | |
| 40 | Comparative | Example 12 | Comparative | 2 | −1 | 2 | 3 | Good | 2.75 | 0.36 | Poor | Poor | Poor |
| | Example 21 | | Example 5 | | | | | | | | | | |
| 41 | Comparative | Comparative | Example 12 | 1 | 2 | 1 | 4 | Good | 1.17 | 0.32 | Poor | Poor | Poor |
| | Example 22 | Example 6 | | | | | | | | | | | |
| 42 | Comparative | Comparative | Example 12 | −2 | −1 | 1 | −2 | Poor | 1.71 | 0.22 | Poor | Good | Poor |
| | Example 23 | Example 7 | | | | | | | | | | | |
| 43 | Comparative | Comparative | Example 12 | −2 | −1 | −1 | −4 | Poor | 3.55 | 0.48 | Poor | Good | Poor |
| | Example 24 | Example 8 | | | | | | | | | | | |
| 44 | Example 20 | Example 10 | Example 12 | 2 | 2 | 2 | 6 | Good | 1.96 | 0.36 | Poor | Good | Poor |
| 45 | Comparative | Example 12 | Comparative | 2 | −1 | −1 | 0 | Poor | 21.15 | 0.44 | Poor | Poor | Poor |
| | Example 25 | | Example 9 | | | | | | | | | | |
| 46 | Comparative | Example 12 | Comparative | 2 | −1 | −1 | 0 | Poor | 28.08 | 0.69 | Poor | Poor | Poor |
| | Example 26 | | Example 10 | | | | | | | | | | |
| 47 | Example 21 | Example 12 | Example 13 | 2 | 2 | 1 | 5 | Good | 15.66 | 0.35 | Poor | Good | Poor |
| 48 | Comparative | Comparative | Example 12 | −2 | −2 | −2 | −6 | Poor | 2.85 | −0.27 | Poor | Good | Poor |
| | Example 27 | Example 11 | | | | | | | | | | | |
| 49 | Comparative | Comparative | Example 12 | −2 | −2 | −2 | −6 | Poor | 4.63 | −0.62 | Poor | Good | Poor |
| | Example 28 | Example 12 | | | | | | | | | | | |
| 50 | Comparative | Example 12 | Comparative | −2 | −1 | −1 | −4 | Poor | 22.10 | 0.77 | Poor | Poor | Poor |
| | Example 29 | | Example 13 | | | | | | | | | | |
| 51 | Comparative | Example 12 | Comparative | −2 | −1 | −1 | 0 | Poor | 26.93 | 0.60 | Poor | Poor | Poor |
| | Example 30 | | Example 14 | | | | | | | | | | |
| 52 | Comparative | Comparative | Example 12 | −2 | 1 | 1 | 0 | Poor | 13.93 | 0.41 | Poor | Good | Poor |
| | Example 31 | Example 15 | | | | | | | | | | | |
| 53 | Comparative | Example 12 | Comparative | 2 | −1 | −1 | 0 | Poor | 26.70 | 0.77 | Poor | Poor | Poor |
| | Example 32 | | Example 16 | | | | | | | | | | |
| 54 | Example 22 | Example 12 | Example 1 | 2 | 2 | 2 | 6 | Good | 2.60 | 0.95 | Good | Good | Good |
| 55 | Example 23 | Example 12 | Example 2 | 2 | 2 | 2 | 6 | Good | 6.12 | 0.73 | Good | Good | Good |
| 56 | Example 24 | Example 12 | Example 3 | 2 | 2 | 2 | 6 | Good | 3.21 | 0.81 | Good | Good | Good |
| 57 | Example 25 | Example 12 | Example 4 | 2 | 1 | 2 | 5 | Good | 13.52 | 0.75 | Good | Good | Good |
| 58 | Example 26 | Example 12 | Example 5 | 2 | 1 | 2 | 5 | Good | 10.58 | 0.76 | Good | Good | Good |
| 59 | Example 27 | Example 12 | Example 6 | 2 | 2 | 2 | 6 | Good | 3.78 | 0.72 | Good | Good | Good |
| 60 | Example 28 | Example 12 | Example 7 | 2 | 2 | 2 | 6 | Good | 3.98 | 0.68 | Good | Good | Good |
| 61 | Example 29 | Example 12 | Example 8 | 2 | 2 | 2 | 6 | Good | 2.20 | 0.69 | Good | Good | Good |
| 62 | Example 30 | Example 12 | Example 9 | 2 | 2 | 2 | 6 | Good | 5.00 | 0.68 | Good | Good | Good |
| 63 | Example 31 | Example 12 | Example 11 | 2 | 1 | 2 | 5 | Good | 13.69 | 0.75 | Good | Good | Good |
| 64 | Example 32 | Example 12 | Example 12 | 2 | 2 | 2 | 6 | Good | 1.00 | 1.00 | Good | Good | Good |
| 65 | Example 33 | Example 12 | Example 14 | 2 | 2 | 2 | 6 | Good | 7.75 | 0.62 | Good | Good | Good |
| 66 | Example 34 | Example 12 | Example 15 | 2 | 2 | 2 | 6 | Good | 7.34 | 0.72 | Good | Good | Good |
| 67 | Example 35 | Example 12 | Example 16 | 2 | 2 | 2 | 6 | Good | 6.54 | 0.59 | Good | Good | Good |
| 68 | Example 36 | Example 12 | Example 17 | 2 | 2 | 2 | 6 | Good | 4.30 | 0.72 | Good | Good | Good |
| 69 | Example 37 | Example 18 | Example 12 | 2 | 2 | 1 | 5 | Good | 1.18 | 0.81 | Good | Good | Good |
| 70 | Example 38 | Example 19 | Example 12 | 2 | 2 | 1 | 5 | Good | 1.75 | 0.75 | Good | Good | Good |

TABLE 7
Preparation Table of Two-Components Dental Resin-Based Cement Composition, and β
value, R² value and Results of Questionary Surveys 2, 3

| | Examples and | Combination of pastes | | Results of questionary survey 2 | | | | | | | Results of questionary survey 3 | | |
|---|---|---|---|---|---|---|---|---|---|---|---|---|---|
| Test | Comparative | | Second | | Kneading | | | Judg- | β | R² | | Ejection | |
| No. | Examples | First paste | paste | Sag | resistance | Admixing | Effects | ment | | | Mixability | properties | Judgment |
| 71 | Comparative | Comparative | Example 15 | 1 | 2 | 2 | 5 | Good | 7.37 | 0.25 | Good | Poor | Poor |
| | Example 33 | Example 1 | | | | | | | | | | | |
| 72 | Comparative | Comparative | Example 15 | −1 | 1 | −1 | −1 | Poor | 11.53 | 0.33 | Good | Poor | Poor |
| | Example 34 | Example 2 | | | | | | | | | | | |
| 73 | Comparative | Comparative | Example 15 | −1 | 1 | −1 | −1 | Poor | 16.79 | 0.26 | Good | Poor | Poor |
| | Example 35 | Example 3 | | | | | | | | | | | |
| 74 | Comparative | Comparative | Example 15 | −1 | −1 | −1 | −3 | Poor | 23.58 | 0.04 | Poor | Poor | Poor |
| | Example 36 | Example 4 | | | | | | | | | | | |
| 75 | Comparative | Comparative | Example 15 | 1 | 2 | −1 | 2 | Good | 2.44 | 0.46 | Poor | Poor | Poor |
| | Example 37 | Example 5 | | | | | | | | | | | |
| 76 | Comparative | Comparative | Example 15 | 1 | 2 | 2 | 5 | Good | 8.02 | 0.39 | Poor | Poor | Poor |
| | Example 38 | Example 6 | | | | | | | | | | | |
| 77 | Comparative | Comparative | Example 15 | −2 | −1 | −1 | −4 | Poor | 12.01 | 0.51 | Poor | Poor | Poor |
| | Example 39 | Example 7 | | | | | | | | | | | |
| 78 | Comparative | Comparative | Example 15 | −2 | −1 | −1 | −4 | Poor | 24.27 | 0.31 | Good | Poor | Poor |
| | Example 40 | Example 8 | | | | | | | | | | | |
| 79 | Example 39 | Example 10 | Example 15 | 1 | 1 | 1 | 3 | Good | 14.55 | 0.30 | Good | Poor | Poor |
| 80 | Comparative | Example 15 | Comparative | 2 | −1 | −2 | −1 | Poor | 2.86 | 1.00 | Poor | Poor | Poor |
| | Example 41 | | Example 9 | | | | | | | | | | |
| 81 | Comparative | Example 15 | Comparative | 2 | −2 | −2 | −2 | Poor | 3.86 | 1.00 | Poor | Poor | Poor |
| | Example 42 | | Example 10 | | | | | | | | | | |
| 82 | Example 40 | Example 15 | Example 13 | 2 | 1 | −1 | 2 | Good | 2.18 | 0.98 | Poor | Poor | Poor |
| 83 | Comparative | Comparative | Example 15 | −2 | −1 | 1 | −2 | Poor | 17.50 | −0.14 | Poor | Poor | Poor |
| | Example 43 | Example 11 | | | | | | | | | | | |
| 84 | Comparative | Comparative | Example 15 | 2 | −1 | −1 | 0 | Poor | 27.77 | −0.26 | Poor | Poor | Poor |
| | Example 44 | Example 12 | | | | | | | | | | | |
| 85 | Comparative | Example 15 | Comparative | 2 | −2 | −2 | −2 | Poor | 2.95 | 0.99 | Poor | Good | Poor |
| | Example 45 | | Example 13 | | | | | | | | | | |
| 86 | Comparative | Example 15 | Comparative | 2 | −2 | −2 | −2 | Poor | 3.83 | 0.96 | Poor | Poor | Poor |
| | Example 46 | | Example 14 | | | | | | | | | | |
| 87 | Comparative | Comparative | Example 15 | 2 | −2 | −2 | −2 | Poor | 92.16 | 0.21 | Poor | Poor | Poor |
| | Example 47 | Example 15 | | | | | | | | | | | |
| 88 | Comparative | Example 15 | Comparative | 2 | −2 | −2 | −2 | Poor | 3.57 | 0.99 | Poor | Poor | Poor |
| | Example 48 | | Example 16 | | | | | | | | | | |
| 89 | Example 52 | Example 1 | Example 15 | 2 | 1 | 2 | 5 | Good | 2.90 | 0.85 | Good | Good | Good |
| 90 | Example 41 | Example 15 | Example 2 | 2 | 1 | 2 | 5 | Good | 1.21 | 0.96 | Good | Good | Good |
| 91 | Example 53 | Example 3 | Example 15 | 2 | 2 | 2 | 6 | Good | 3.28 | 0.97 | Good | Good | Good |
| 92 | Example 42 | Example 15 | Example 4 | 2 | 1 | 2 | 5 | Good | 1.81 | 0.99 | Good | Good | Good |
| 93 | Example 43 | Example 15 | Example 5 | 2 | 1 | 2 | 5 | Good | 1.41 | 0.99 | Good | Good | Good |
| 94 | Example 44 | Example 6 | Example 15 | 2 | 2 | 2 | 6 | Good | 1.95 | 1.00 | Good | Good | Good |
| 95 | Example 45 | Example 7 | Example 15 | 2 | 2 | 2 | 6 | Good | 1.81 | 0.99 | Good | Good | Good |
| 96 | Example 46 | Example 8 | Example 15 | 2 | 2 | 2 | 6 | Good | 3.28 | 1.00 | Good | Good | Good |
| 97 | Example 47 | Example 9 | Example 15 | 2 | 2 | 2 | 6 | Good | 1.44 | 0.99 | Good | Good | Good |
| 98 | Example 48 | Example 15 | Example 11 | 2 | 2 | 2 | 6 | Good | 1.84 | 1.00 | Good | Good | Good |
| 99 | Example 55 | Example 12 | Example 15 | 2 | 2 | 2 | 6 | Good | 7.34 | 0.72 | Good | Good | Good |
| 100 | Example 48 | Example 15 | Example 14 | 2 | 2 | 2 | 6 | Good | 1.09 | 0.98 | Good | Good | Good |
| 101 | Example 49 | Example 15 | Example 15 | 2 | 2 | −1 | 3 | Good | 1.00 | 1.00 | Good | Good | Good |
| 102 | Example 50 | Example 15 | Example 16 | 2 | 2 | 1 | 5 | Good | 1.11 | 0.95 | Good | Good | Good |
| 103 | Example 51 | Example 17 | Example 15 | 2 | 2 | 2 | 6 | Good | 1.71 | 1.00 | Good | Good | Good |
| 104 | Example 56 | Example 18 | Example 15 | 2 | 2 | 2 | 6 | Good | 9.28 | 0.87 | Good | Good | Good |
| 105 | Example 57 | Example 19 | Example 15 | 2 | 2 | 2 | 6 | Good | 14.30 | 0.97 | Good | Good | Good |
| 106 | Example 58 | Example 18 | Example 4 | 2 | 2 | −1 | 3 | Good | 16.90 | 0.94 | Poor | Poor | Poor |
| 107 | Example 59 | Example 19 | Example 4 | 2 | 1 | −1 | 2 | Good | 26.10 | 0.98 | Poor | Good | Poor |
| 108 | Example 60 | Example 19 | Example 5 | 2 | 1 | −1 | 2 | Good | 20.40 | 0.98 | Poor | Poor | Poor |
| 109 | Example 61 | Example 18 | Example 10 | 2 | 2 | −1 | 3 | Good | 17.20 | 0.93 | Poor | Poor | Poor |
| 110 | Example 62 | Example 19 | Example 10 | 2 | 1 | −1 | 2 | Good | 26.59 | 0.98 | Poor | Poor | Poor |

Taking notice of the test results of Examples 20, 21, 39 and 40, it was found that, when using first and second pastes satisfying the constituent features (a) to (c) and (e) in combination, one paste having a yield viscosity (d) of 100 to 3,000 [Pa·s], the other paste having a yield viscosity of 70 to 4,000 [Pa·s], good survey results of the operability as a hand kneading type supply system are obtained and these composition systems can be employed.

Test Nos. 106 to 110 describe the results of the test in which the survey 2 and survey 3 were carried out as the evaluation of operability of two-component dental resin-based cement compositions using the pastes of Examples 4, 5, 11, 18 and 19 in combination.

Since the combination of pastes of two-component resin-based cement composition in Examples 58 to 62 is a dental resin-based cement composition whose operability of “sagging”, “kneading resistance” and “admixing” was judged good in the evaluation 1, all results of the survey 2 was good. However, as is apparent from the survey 3, even when operability as a hand kneading type supply system which is a conventional method is good, mixability or ejecting properties of an automix container as a comparatively new supply system is sometimes inferior. When the automix container is used, the coefficient β and R² are more preferably within a predetermined range.

The dental resin-based cement composition and the method for controlling flow properties of the same of the present invention can provide a dental resin-based cement composition having excellent operability which can contribute to shortening of an operation time and reduction of technical errors.

What is claimed is:

1. A dental resin cement composition comprising at least two kinds of pastes, one kind of a paste comprising (a) 20 to 50 parts by weight of a polymerizable monomer composition and (b) 50 to 80 parts by weight of a filler consisting of an inorganic compound, or an organic composite comprising an inorganic compound, amounts of (a) and (b) being based on conversion of 100 parts by weight of the dental resin-based cement composition paste, and the filler (b) comprising (c) 2.0 to 10.0 parts by weight of fine silica particles based on the above conversion, and having (d) a yield viscosity of from 100 to 3,000 [Pa·s], and (e) a thixotropy index of 3.0 or more; and another kind of a paste having the same constituent features (a) to (c) and (e) except that the paste has (d) a yield viscosity of from 70 to 4,000 [Pa·s], wherein upon use, the at least two kinds of pastes are used by mixing them.

2. The dental resin-based cement composition according to claim 1, wherein the composition comprises at least two kinds of pastes, each of the pastes comprising (a) 20 to 50 parts by weight of a polymerizable monomer composition and (b) 50 to 80 parts by weight of a filler consisting of an inorganic compound, or an organic composite comprising an inorganic compound, amounts of (a) and (b) being based on conversion of 100 parts by weight of the dental resin-based cement composition paste, and the filler (b) comprising (c) 2.0 to 10.0 parts by weight of fine silica particles based on the above conversion, and having (d) a yield viscosity of from 100 to 3,000 [Pa·s], and (e) a thixotropy index of 3.0 or more, and wherein upon use, the at least two kinds of pastes are used by mixing them.

3. The dental resin-based cement composition according to claim 1 or 2, wherein the composition further includes a characteristic (f) in the two kinds of pastes according to claim 1, letting the paste having the smaller yield viscosity (d) to be a first paste, and the other paste to be a second paste, 20 viscosity values are determined by using 20 measurement points within the range from 0.1 [s⁻¹] to 10 [s⁻¹] so as to include shear rates 0.1 [s⁻¹] and 10 [s⁻¹] and to make the intervals between logarithms of the shear rates equal, and when a proportionality constant β and a determination coefficient R² of a linear approximation curve passing through the origin are calculated with respect to a function in which 20 viscosity values of the first paste are independent variables and 20 viscosity values of the second paste are dependent variables, β is within the range from 1.00 to 15.00 and R² is within the range from 0.50 to 1.00.

4. A method for controlling the flow properties of a dental resin-based cement composition, which comprises mixing at least two kinds of pastes, each of the pastes comprising (a) 20 to 50 parts by weight of a polymerizable monomer composition and (b) 50 to 80 parts by weight of a filler consisting of an inorganic compound or an organic composite comprising an inorganic compound, amounts of (a) and (b) being based on conversion of 100 parts by weight of the dental resin-based cement composition paste, and the filler (b) comprising (c) 2.0 to 10.0 parts by weight of fine silica particles based on the above conversion, and using the mixture as a dental resin-based cement composition,
wherein upon use, one kind of the pastes having (d) a yield viscosity of 100 to 3,000 [Pa·s] and (e) a thixotropy index of 3.0 or more, and another kind of the pastes having (d) a yield viscosity of 70 to 4,000 [Pa·s], and the same (e) as that described above are selected.

5. The method according to claim 4, wherein the method comprises mixing at least two kinds of pastes, each comprising (a) 20 to 50 parts by weight of a polymerizable monomer composition and (b) 50 to 80 parts by weight of a filler consisting of an inorganic compound, or an organic composite comprising an inorganic compound, amounts of (a) and (b) being based on conversion of 100 parts by weight of a dental resin-based cement composition, and the (b) filler comprising (c) 2.0 to 10.0 parts by weight of silica fine particles, and using the mixture as a dental resin-based cement composition,
where upon use, the at least two kinds of pastes, each having (d) a yield viscosity of 100 to 3,000 [Pa·s] and (e) a thixotropy index of 3.0 or more, are selected.

6. The method according to claim 4 or 5, wherein as the two kinds of pastes as defined in claim 3, the two kinds of pastes are selected so that (f) letting the paste having the smaller yield viscosity (d) to be a first paste and the other paste to be a second paste, 20 viscosity values are determined by using 20 measurement points within the range from 0.1 [s⁻¹] to 10 [s⁻¹] so as to include shear rates 0.1 [s⁻¹] and 10 [s⁻¹] and to make the intervals between logarithms of the shear rates equal, and when a proportionality constant β and a determination coefficient R² of a linear approximation curve through the origin are calculated with respect to a function in which 20 viscosity values of the first paste are independent variables and 20 viscosity values of the second paste are dependent variables, β is within the range from 1.00 to 15.00 and R² is within the range from 0.50 to 1.00.
